(19) United States
(12) Patent Application Publication
(10) Pub. No.: US 2008/0207673 A1
(43) Pub. Date: Aug. 28, 2008

(54) Method for Treating Cancer, Coronary, Inflammatory and Macular Disease, Combining the Modulation of Zinc-and/or Copper Dependent Proteins

(76) Inventor: Michel Xilinas, Aradippou (CY)

Correspondence Address: BROWDY AND NEIMARK, P.L.L.C.;624 NINTH STREET, NW, SUITE 300, WASHINGTON, DC, 20001-5303, US

(21) Appl. No.: 11/913,610

(22) PCT Filed: May 4, 2006
(86) PCT No.: PCT/IB06/01149

§ 371 Date: Nov. 5, 2007

(30) Foreign Application Priority Data
May 4, 2005 (CY) 05/00027
May 9, 2005 (CY) 05/00029

(51) Int. Cl. A61K 31/4745; A61K 31/47; A61P 35/00; A61P 35/04

(52) U.S. Cl. 514/292; 514/311

(57) ABSTRACT

A method for treating diseases selected among cancer, coronary disease, inflammatory disease and macular disease by chelation therapy is disclosed.

The present invention relates to a new use of chelating agents that chelate strongly copper and zinc ions in different tissues and cells. Especially, the invention pertains to the use of clioquinol and phanquinone in combination or separately, for the manufacture of a pharmaceutical composition for the treatment, modulation or prevention of pathological conditions related to proteins interfering in the pathology of cancer, atherosclerotic plaque rupture, auto-immune conditions, macular degeneration and of neoangiogenesis secondary to different pathological conditions.

### BACKGROUND FOR THE INVENTION

Zinc and copper interfere in many physiological enzymatic systems having co-factor, modulating or catalytic effect. The metabolism of both minerals is in equilibrium allowing a steady state within the body under normal conditions. In several diseases zinc and copper levels are modified within the process of the pathogenesis of the abnormal condition. In cancer disease in general the zinc and copper are involved in metalloprotein reactions of neongiogenesis. In rheumatic disease zinc and copper are involved in the pathology of the auto-immune conditions. Acute coronary syndromes are related with phenomena on the atheromatic plaque that is disrupted and ruptured and where zinc plays a major role.

Zinc plays a critical role in many biochemical functions involving protein and nucleic acid metabolism. Zinc serves as a catalytic agent for over 200 enzymes as well as a structural agent for various proteins, hormones and nucleotides. Zinc body stores amount to about 2-2.5 g, of which 60 percent is found in muscle and 20-30 percent in bone. Zinc rich foods include seafood, meat, nuts, and milk. Diets high in phytate fibre are associated with a low bioavailability of dietary zinc. Zinc is required for normal olfactory and taste acuity.

Dietary zinc deficiency stimulates the production of hydrogen peroxide and free radical production, causing increased DNA damage because of an increased rate of oxidative damage and/or a reduction in DNA repair.

Copper is an essential trace element, which is distributed throughout the body. Most people get only about 1 mg of copper a day in the diet; compared to the recommended 1.5 to 3.0 mg. Copper-rich foods include beans, nuts, and seafood. Copper deficiency is rare. Oral administration of large doses of zinc or iron induces signs of copper deficiency. Commonly used indices of copper sufficiency are plasma or serum copper, ceruloplasmin and erythrocyte superoxide dismutase (Cu—Zn SOD). Normal plasma or whole blood copper levels range from 13 to 22 μmol/L. Plasma copper levels increase with increasing age.

Heizmann and Cox report that S100 proteins (16 members) show a very divergent pattern of cell- and tissue-specific expression, of subcellular localizations and relocations, of post-translational modifications, and of affinities for Ca2+, Zn2+, and Cu2+, consistent with their pleiotropic intra- and extracellular functions. Up to 40 target proteins are reported to interact with S100 proteins and for S100A1 alone 15 target proteins are presently known. Therefore it is not surprising that many functional roles have been proposed and that several human disorders such as cancer, neurodegenerative diseases, cardiomyopathies, inflammations, diabetes, and allergies are associated with an altered expression of S100 proteins. Despite the numerous putative functions of S100 proteins, their three-dimensional structures of, e.g., S100B, S100A6, and S100A7 are surprisingly similar. (Heizmann C W, Cox J A. New perspectives on S100 proteins: a multi-functional Ca(2+)-, Zn(2+)- and Cu(2+)-binding protein family. Biometals. 1998; 11:383-97).

Copper is the essential redox-active center and a key component of lysyl oxidase (maintains connective tissue integrity through cross-linking of elastin and collagen), cytochrome c oxidase (electron transport chain), ceruloplasmin (ferroxidase activity), superoxide dismutase (free radical detoxification), tyrosinase, ascorbate oxidase and dopamine-hydroxylase (catechol production).

Copper, zinc and manganese comprise three forms of superoxide dismutase (SOD): intracellular CuZnSOD and MnSOD and extracellular SOD. Intracellular CuZnSOD comprises about 85-90 percent of total cellular SOD activity, the majority of which resides in peroxisomes. MnSOD comprises about 10-15 percent of total cellular SOD and is located in mitochondria. Copper is found in the cell nucleus, closely associated with chromosomes and the DNA base guanine. DNA-associated copper has been suggested to be involved in maintaining normal chromosome structure and in gene regulation.

Another very interesting area of research into CuZnSOD is being done in the area of Alzheimer's disease. The brain lesions that are characteristic of Alzheimer's disease show evidence of oxidative processes as well as other damage, such as inflammation. The zinc-deficient CuZnSOD not only did not function as an antioxidant, but instead behaved like a pro-oxidant compound. It was observed that the loss of zinc from CuZnSOD, which still had its copper, was sufficient to induce apoptosis in cultured motor neurons, This finding is significant not only in the disease of ALS, which has been directly tied to this enzyme, but also to Alzheimer's disease, where pathological behaviour of this enzyme is suspected.

Activation of macrophages leads to the secretion of cytokines and enzymes that shape the inflammatory response and increase metabolic processes. This, in turn, results in increased production of reactive oxygen species. The role of Cu/Zn superoxide dismutase (SOD-1), an important enzyme in cellular oxygen metabolism, was examined in activated peritoneal elicited macrophages (PEM) and in several inflammatory processes in vivo. LPS and TNF-alpha induced SOD-1 in PEM. SOD-1 induction by LPS was mainly via extracellular signal-regulated kinase-1 activation. Transgenic mice overexpressing SOD-1 demonstrated a significant increase in the release of TNF-alpha and of the metalloproteinases MMP-2 and MMP-9 from PEM. Disulfiram (DSF), an inhibitor of SOD-1, strongly inhibited the release of TNF-alpha, vascular endothelial growth factor, and MMP-2 and MMP-9 from cultured activated PEM. These effects were prevented by addition of antioxidants, further indicating involvement of reactive oxygen species. In vivo, transgenic mice overexpressing SOD-1 demonstrated a 4-fold increase in serum TNF-alpha levels and 2-fold stronger delayed-type hypersensitivity reaction as compared with control nontransgenic mice. Conversely, oral administration of DSF lowered TNF-alpha serum level by 4-fold, lowered the delayed-type hypersensitivity response in a dose-dependent manner, and significantly inhibited adjuvant arthritis in Lewis rats. The data suggest an important role for SOD-1 in inflammation, establish DSF as a potential inhibitor of inflammation, and raise the possibility that regulation of SOD-1 activity may be important in the treatment of immune-dependent pathologies. (Marikovsky M, Ziv V, Nevo N, Harris-Cerruti C, Mahler O. Cu/Zn superoxide dismutase plays important role in immune response, J Immunol; 170:2993-3001).

Activation of macrophages leads to the secretion of cytokines and enzymes that shape the inflammatory response and increase metabolic processes. This, in turn, results in increased production of reactive oxygen species. The role of Cu/Zn superoxide dismutase (SOD-1), an important enzyme in cellular oxygen metabolism, was examined in activated peritoneal elicited macrophages (PEM) and in several inflammatory processes in vivo. The data provided by Marikovsky et al, suggest an important role for SOD-1 in inflammation, establish disulfiram, an inhibitor of SOD-1, as a potential inhibitor of inflammation, and raise the possibility that regulation of SOD-1 activity may be important in the treatment of immune-dependent pathologies. (Marikovsky M, Ziv V, Nevo N, Harris-Cerruti C, Mahler O. Cu/Zn superoxide dismutase plays important role in immune response. J Immunol, 2003; 170:2993-3001).

Copper ions stimulate proliferation of human umbilical artery and vein endothelial cells but not human dermal fibroblasts or arterial smooth muscle cells. Incubation of human umbilical vein endothelial cells for 48 h with 500 μM CuSO₄ in a serum-free medium in the absence of exogenous growth factors results in a twofold increase in cell number, similar to the cell number increase induced by 20 ng/ml of basic fibroblast growth factor under the same conditions. Copper-induced proliferation of endothelial cells is not inhibited by 10% fetal bovine serum or by the presence of antibodies against a variety of angiogenic, growth, and chemotactic factors including angiogenin, fibroblast growth factors, epidermal growth factor, platelet-derived growth factor, tumor necrosis factor-alpha, transforming growth factor-beta, macrophage/monocyte chemotactic and activating factor, and macrophage inflammatory protein-1alpha. Moreover, despite the previous observations that copper increased total specific binding of 125I-angiogenin to endothelial cells, binding to the 170 kDa receptor is not changed; hence, the mitogenic activity of angiogenin is not altered by copper. Copper-induced proliferation, along with early reports that copper induces migration of endothelial cells, may suggest a possible mechanism for the involvement of copper in the process of angiogenesis. (Hu G F. Copper stimulates proliferation of human endothelial cells under culture. J Cell Biochem. 1998; 69:326-35).

Copper is an essential trace element for proper functioning of the immune system. A diet deficient in copper affects the human immune system, reducing the activity of some cells that attack invading bacteria. Copper deficiency in humans is associated with altered bone marrow white cell maturation, neutropenia, and an altered B lymphocyte antibody response. Phagocytosis by neutrophils is associated with production of oxygen radical species, which are inactivated by several enzymes, including CuZnSOD.

The extracellular matrix (ECM) of the simple multicellular organism Volvox contains many region-specific morphological elements and mediates a variety of developmental and physiological responses by modification of its components. The fact that >95% of the mature organism is ECM makes Volvox suitable as a model system for ECM investigations. VMPs are a family of Volvox genes that are homologous to zinc-dependent matrix metalloproteinases (MMPs). Heitzer et al described the identification and purification of the first VMP protein, VMP3. The 470-kDa VMP3 glycoprotein is localized within the ECM, and its biosynthesis is induced by the sex pheromone. The metal binding motif of VMP3 is QEXXH, not HEXXH as known for approximately 1300 other metalloproteinases. VMP3 shows proteinase activity and is inhibited by EDTA or the MMP inhibitor GM 6001, but in contrast to all known proteinases, VMP3 clearly prefers copper for activity rather than zinc. The exchange from Q to H within the QEXXH motif abolishes its copper preference. The unique properties of VMP3 suggest a novel type of metalloproteinase, (Heitzer M, Hallmann A. An extracellular matrix-localized metalloproteinase with an exceptional QEXXH metal binding site prefers copper for catalytic activity. J Biol. Chem. 2002 2; 277:28280-6).

Glycyl-histidyl-lysine-Cu2+ (GHK-Cu) is a tripeptide-copper complex known to be a potent wound healing agent. Simeon et al previously showed its ability to stimulate in vitro and in vivo the synthesis of extracellular matrix components. The aim of the study of Simeon et al was to determine the effects of GHK-Cu on MMP-2 synthesis by dermal fibroblasts in culture. Simeon et al showed that GHK-Cu increased MMP-2 levels in conditioned media of cultured fibroblasts. This effect was reproduced by copper ions but not by the tripeptide GHK alone. This stimulation was accompanied by an increase of MMP-2 mRNA level. We also showed that GHK-Cu increased the secretion of the tissue inhibitors of metalloproteinases, TIMP-1 and TIMP-2. Taken together, the results of this study show that GHK-Cu is not only an activator of connective tissue production but also of the remodeling of the extracellular matrix. It is able to modulate MMP expression by acting directly on wound fibroblasts. (Simeon A, Emonard H. Hornebeck W, Maquart F X. The tripeptide-copper complex glycyl-L-histidyl-L-lysine-Cu2+ stimulates matrix metalloproteinase-2 expression by fibroblast cultures. Life Sci. 2000 Sep 22; 67(18):2257-65).

NO is an endogenous signalling molecule that is synthesized from L-arginine and O₂ by a family of NO syntheses (NOS's) that includes neuronal, inducible, and endothelial NOS (nNOS, iNOS, and eNOS, respectively). NOS maintains two catalytic domains that consist of a C-terminal reductase where NADPH, FMN, and FAD bind, and an N-terminal oxygenase domain where heme, 5,6,7,8-tetrahydrobiopterin (BH₄), oxygen, and L-arginine bind. The catalytic mechanisms of NOS involve flavin-mediated electron transport from C-terminal-bound NADPH to the N-terminal heme center, where oxygen is reduced and incorporated into the guanidine group of L-arginine, giving rise to NO and L-citrulline.

All three NOS's are dimeric enzymes comprised of two identical subunits, and NOS is catalytically active only in dimeric form. X-ray crystallography for all three isoforms of NOS shows a zinc thiolate (ZnS₄) cluster formed by a zinc ion coordinated in a tetrahedral conformation with pairs of symmetrically oriented and phylogenetically conserved cysteine residues at the dimer interface. Mutation within a C(X₄)C motif prevents the binding of zinc, BH₄, or L-arginine and eliminates enzyme activity, suggesting that stabilization of the dimer interface by the zinc-thiolate center is key for catalytic activity. Regulation of NOS subunit interactions could, therefore, provide a mechanism for modulation of enzyme activity in vivo, (Ming-Hui Zou, Chaomei Shi and Richard A. Cohen Oxidation of the zinc-thiolate complex and uncoupling of endothelial nitric oxide synthase by peroxynitrite 3 Clin Invest 2002; 109: 817-826).

Nitric oxide (NO) and reactive oxygen species (ROS) are emerging as important regulators of angiogenesis. NO enhances vascular endothelial growth factor (VEGF) synthesis in several cell types and is required for execution of VEGF angiogenic effect in endothelial cells. Similarly, hydrogen peroxide induces VEGF synthesis and recent studies indicate the involvement of ROS in signaling downstream of VEGF stimulation. VEGF synthesis can not only be enhanced by gene transfer of VEGF but also by overexpression of NO synthase genes. The examination of the possibility of augmentation of VEGF production by gene transfer of copper/zinc superoxide dismutase (CuZnSOD, SOD1). Overexpression of human SOD1 in mouse NIH 3T3 fibroblasts increased SOD activity, enhanced intracellular generation of H₂O₂ and significantly stimulated VEGF production as determined by increase in VEGF promoter activity, VEGF mRNA expression and VEGF protein synthesis. The stimulatory effect on VEGF synthesis induced by SOD1 gene transfer was reverted by overexpression of human catalase. The effect of H₂O₂ produced by engineered cells is mediated by activation of hypoxia-inducible factor response element (HRE) as well as Sp1 recognition site of VEGF promoter. This data suggest the feasibility of stimulation of angiogenesis by overexpression of SOD1 (Grzenkowicz-Wydra J, Cisowski 3, Nakonieczna 3, Zarebski A, Udilova N, Nohl H, Jozkowicz A, Podhajska A, Dulak 3. Gene transfer of CuZn superoxide dismutase enhances the synthesis of vascular endothelial growth factor. Mol Cell Biochem. 2004; 264:169-81).

Byrnes et al describe that mechanistic details of the interaction of 1,10-phenanthroline and its copper complex with Ehrlich ascites tumor cells were examined, using inhibition of cell proliferation, DNA breakage, and increased membrane permeability as indices of cellular damage. The metal chelating agent, 1,10-phenanthroline (OP), the 1:0.5 complex of 1,10-phenanthroline and CuCl₂ [(OP)₂Cu], and CuCl₂ inhibited growth of Ehrlich ascites tumor cell monolayers during 48-h treatments by 50% at about 3.5, 2, and 70 nmol/10⁵ cells/mL, respectively. (OP)₂Cu at 10 nmol/10⁵ cells also enhanced uptake of trypan blue dye during 6 h of treatment, while dye uptake in OP- and CuCl₂-treated cells remained similar to controls. DNA breakage, measured by DNA alkaline elution, was produced during 1-h treatments with (OP)₂Cu at drug/cell ratios similar to those producing growth inhibition. They conclude that multiple mechanisms for generation of oxidative damage occur in (OP)₂Cu-treated cells and that growth inhibition produced by OP or (OP)₂Cu, as well as the low levels of strand scission produced by OP, was not reversed by scavengers. (Byrnes R W, Antholine W E, Petering D H. Interactions of 1,10-phenanthroline and its copper complex with Ehrlich cells. Free Radic Biol Med. 1992; 12:457-69).

Reactive oxygen species (ROS) are implicated in reperfusion injury after focal cerebral ischemia (FCI). Reactive oxygen species regulate activity of transcription factors like NF-kappaB. The authors investigated the role of ROS in NF-kappaB activity after FCI using transgenic mice that overexpressed human copper/zinc-superoxide dismutase (SOD1) and that had reduced infarction volume after FCI. The current findings of Huang et al provide the first evidence that SOD1 overexpression attenuates activation of NF-kappaB after transient FCI in mice and that preventing this early activation may block expression of downstream deleterious genes like c-myc, thereby reducing ischemic damage. (Huang C Y, Fujimura M, Noshita N. Chang Y Y, Chan P H. SOD1 down-regulates NF-kappaB and c-Myc expression in mice after transient focal cerebral ischemia. J Cereb Blood Flow Metab. 2001; 21:163-73).

Angiogenesis is the growth of new vessels from pre-existing blood vessels. Angiogenesis is critical during embryogenesis but occurs minimally in healthy adults, except in wound repair, inflammation, female reproductive organs, and pathologic conditions. Various growth factors and proteins, elements of the extracellular matrix, components of the coagulation/fibrinolytic system, and platelets interact with the endothelial cells and pericytes of blood vessels to regulate angiogenesis. Characterization of angiogenic factors has revealed that remodelling of the extracellular matrix occurs during angiogenesis, mediated by integrins that are found on the endothelial cell surface membrane. Counter-regulatory antiangiogenic proteins and molecules that show an intricate balance in the regulation of angiogenesis have also been characterized. Components of the coagulation/fibrinolysis cascade also play a critical role in angiogenesis. Elucidation of the mechanisms of angiogenesis has led to better understanding of certain disease states.

The importance of copper is also underscored in cancer. The underlying hypothesis of antiangiogenesis using copper-reduction therapy is that the level of copper required for angiogenesis is higher than that required for essential copper-dependent cellular functions. The assumption is that there is a window of copper deficiency in which angiogenesis is impaired, but other copper-dependent cellular processes are not affected enough to cause clinical toxicity. Unlike other anti-cancer agents now being studied around the world, copper-reduction therapy is not limited to a single type of cancer. The relationship of copper to cancer is not causative but associative. Cancer cells in a high copper environment find it easy to proliferate into tumours. In an environment low in copper, cancer cells would remain dormant or very slow-growing, increasing survival time.

Although chelation therapy has been used since the 1940's for a wide variety of ailments, it is still considered “alternative medicine” for anything other than heavy metal poisoning. Chelation therapy usually consists of an intravenous solution containing a synthetic amino acid called ethylene diamine-tetraacetic acid (EDTA). When administered properly, it is a safe and effective way to deplete heavy metals and other toxins from the bloodstream. Although the authors could not locate any study results using EDTA chelation for copper-reduction therapy, one lymphoma patient known to them did make the attempt. Standard EDTA chelation administrations were used for five treatments, but the therapy abandoned when test results indicated it was ineffective in lowering copper levels. (http://www.coldcure.com/html/anti_ang.html).

The Investigational Board Approval (IRB) Submission is centered on antiangiogenesis and antioxidant treatments diminishing tumor growth and metastasis, specifically using tetrathiomolybdate, zinc, ascorbic acid, N-acetylcysteine and vitamin B6. Copper bound to ceruloplasmin increases angiogenic activity and correlates with tumour incidence, burden and malignant progression. Copper has been found to behave as a molecular switch for activating cytokines, interleukin-1 (IL-1) and tumour necrosis factor-alpha (TNF-a), and growth factors such as basic fibroblast growth factor (bFGF) and vascular endothelial growth factor (VEGF). All four of the above signalling factors have been shown to be angiogenic. Copper seems to act as an “obligatory cofactor” allowing for the angiogenic activator to become functional. In addition, copper was found to stimulate the directional migration of endothelial cells where other trace metals were not. The underlying hypothesis of antiangiogenesis using copper-reduction therapy is that the level of copper required for angiogenesis is higher than that required for essential copper-dependent cellular functions. Having established that copper is intimately involved in tumor growth via the angiogenic pathway, it is feasible to propose a method of treatment, which will decrease the body's concentration of copper. (http://www.cancerprotocol.com/copperprotocol.html).

Angiogenesis is now recognized as a crucial process in tumour development, including hepatocellular carcinoma (HCC). Since HCC is known as a hypervascular tumour, anti-angiogenesis is a promising approach to inhibit the HCC development. Yoshii et al examined the effect of Cu-chelating Their results suggested that Cu plays a pivotal role in tumour development and angiogenesis in the murine HCC cells, and Cu-chelators, could inhibit angiogenesis and enhance apoptosis in the tumour with consequent suppression of the tumour growth in vivo. (Yoshii 3, Yoshiji H, Kuriyama S, Ikenaka Y, Noguchi R, Okuda H. Tsujinoue H, Nakatani T. Kishida H, Nakae D, Gomez D E, De Lorenzo M S, Tejera A M, Fukui H. The copper-chelating agent, trientine, suppresses tumour development and angiogenesis in the murine hepatocellular carcinoma cells. Int J Cancer. 2001; 94:768-73).

Copper plays an essential role in promoting angiogenesis. Tumours that become angiogenic acquire the ability to enter a phase of rapid growth and exhibit increased metastatic potential, the major cause of morbidity in cancer patients. Pan reported that copper deficiency induced by Tetrathiomolybdate™ significantly impairs tumour growth and angiogenesis in two animal models of breast cancer: an inflammatory breast cancer xenograft in nude mice and Her2/neu cancer-prone transgenic mice. (Pan Q, Kleer C G, van Golen K L, Irani J, Bottema K M, Bias C, De Carvalho M, Mesri E A, Robins D M, Dick R D, Brewer G J, Merajver S D. Copper deficiency induced by tetrathiomolybdate suppresses tumor growth and angiogenesis. Cancer Res. 2002; 62:4854-9).

A growing arena of cancer research involves the inhibition of angiogenesis. Cancer cells require food, oxygen and growth proteins in order to grow and spread. These essential nutrients are transported to the cancer cells by blood vessels. Angiogenesis is the process of creating new blood vessels necessary to transport nutrients, oxygen, hormones and proteins to the cancer cells. Two of several key proteins that are necessary for the process of angiogenesis are called vascular endothelial growth factor (VEGF) and matrix metalloproteinases (MMPs). VEGF causes endothelial cells (cells comprising the innermost layer of blood vessels) to replicate and migrate from existing blood vessels to the cancer. Endothelial cells secrete MMPs, which create an opening in existing tissues surrounding the cancer, allowing the endothelial cells to move near the cancer and form new blood vessels to feed the cancer. Researchers have been evaluating targeted treatment approaches, which hinder or reduce the effects of VEGF and thus, slow cancer progress.

Daniel et al (2005) have reported that a unique feature of cancer cells is to accumulate high concentrations of copper. The authors believe that a potential strategy for cancer chemotherapy could involve the use of organic ligands that act as copper sensors and bind with elevated copper in cancer cells and tissues. These complexes would act as proteosome inhibitors and apotosis inducers to tumour cells. Because normal cells contain only trace amounts of copper, the organic ligands should form far fewer complexes with copper int hem, thus exposing the normal cells to a minimal dose and reducing toxicity. They propose that treatment with copper-binding compounds such as CQ and PDTC will result in these compounds behaving as tumor ‘sensors’ using copper as a selection criterion. Therefore, this application showing that effectively clioquinol may convert the proangiogenic co-factor into a cancer-specific killing agent. (Daniel K G, Chen D F, Orlus, Cui Q C, Miller F R, Dou Q P. Clioquinol and pyrrolidine dithiocarbamate complex with copper to form proteasome inhibitors and apoptosis inducers in human breast cancer cells. Breast Cancer Res. 2005; 7:R897-908.

Fujiwara et al investigated the effect of zinc sulfate on the proliferation of cultured bovine aortic smooth muscle cells stimulated with or without growth factors. It was shown that stimulation of the [3H]thymidine incorporation by either basic or acidic fibroblast growth factor was significantly potentiated by zinc. Their data suggest that zinc is a particular heavy metal, which potentiates vascular smooth muscle cell proliferation stimulated by basic and acidic fibroblast growth factors as well as thrombospondin. They conclude that zinc may be involved in the intimal hyperplasia of atherosclerosis. (Fujiwara Y, Kaji T. Zinc potentiates the stimulation by basic and acidic fibroblast growth factors on the proliferation of cultured vascular smooth muscle cells Res Commun Mol Pathol Pharmacol. 1997; 97:95-106).

Deregulated extracellular matrix (FCM) metabolism may contribute to vascular remodeling during the development and complication of human atherosclerotic lesions. Galls et al investigated members of all three MMP classes (interstitial collagenase, MMP-1; gelatinases, MMP-2 and MMP-9; and stromelysin, MMP-3) and their endogenous inhibitors (TIMPs 1 and 2) by immunocytochemistry, zymography, and immunoprecipitation. Normal arteries stained uniformly for 72-kD gelatinase and TIMPs. The MMP inhibitors, EDTA and 1,10-phenanthroline, as well as recombinant TIMP-1, reduced these activities which co localized with regions of increased immunoreactive MMP expression, i.e., the shoulders, core, and microvasculature of the plaques. Focal overexpression of activated MMP may promote destabilization and complication of atherosclerotic plaques and provide novel targets for therapeutic intervention. (Galis Z S, Sukhova G K, Lark M W, Libby P. Increased expression of matrix metalloproteinases and matrix degrading activity in vulnerable regions of human atherosclerotic plaques. J Clin Invest. 1994; 94:2493-503).

Oxidation of low density lipoproteins (LDL) in blood vessel walls plays a significant role in the development of atherosclerosis. LDL oxidation in vitro is greatly accelerated by the presence of “catalytic” iron or copper ions, which have already been shown to be present within advanced atherosclerotic lesions. Evans et al demonstrated that mechanical damage to human arterial wall samples (both normal and early or intermediate atherosclerotic lesions) causes release of “catalytic” iron and copper ions, to an extent increasing with the damage. It may be that traumatic (e.g. during angioplasty) or other injury to the vessel wall contributes to the generation of metal ions that can facilitate LDL oxidation and other free radical reactions, so promoting atherosclerosis. (Evans P J, Smith C, Mitchinson M J, Halliwell B. Metal ion release from mechanically-disrupted human arterial wall. Implications for the development of atherosclerosis. Free Radic kes. 1995; 23:465-9).

Lipid peroxidation within human arterial lesions is thought to play an important role in the development of atherosclerosis. Peroxidation can be accelerated by the presence of ‘catalytic’ iron or copper ions. Gruel samples from advanced atherosclerotic lesions in the abdominal aortae of human cadavers were tested by Smith et al. for pro-oxidant properties. All samples contained bleomycin-detectable iron and phenanthroline-detectable copper. Almost all gruel samples stimulated peroxidation of rat liver microsomes, and this was usually inhibited by the iron-ion chelator desferrioxamine. Some samples stimulated formation of hydroxyl radicals from H₂O₂ in the presence of ascorbate, a reaction again inhibited by desferrioxamine, Smith et al conclude that the interior of human advanced atherosclerotic lesions is a highly pro-oxidant environment, and that the use of copper or iron ions to promote peroxidation of low-density lipoproteins in vitro may be a valid model for events in the arterial wall. (Smith C, Mitchinson M J, Aruoma O I, Halliwell B. Stimulation of lipid peroxidation and hydroxyl-radical generation by the contents of human atherosclerotic lesions. Biochem J. 1992; 286:901-5).

Endothelial adhesion molecule expression and monocyte recruitment are causal events in human atherosclerosis, and are believed to be caused, in part, by oxidative stress. Zhang et al suggested that intracellular, but not extracellular, transition metal ions mediate inflammatory cytokine-induced SP-1 activation and adhesion molecule expression in endothelial cells. (Zhang W J, Frei B. Intracellular metal ion chelators inhibit TNFalpha-induced SP-1 activation and adhesion molecule expression in human aortic endothelial cells. Free Radic Biol Med. 2003; 34:674-82).

The induction of an acute inflammatory response followed by the release of polypeptide cytokines and growth factors from peripheral blood monocytes has been implicated in mediating the response to vascular injury. Because the Cu2+-binding proteins interleukin-1alpha and fibroblast growth factor 1 are exported into the extracellular compartment in a stress-dependent manner by using intracellular Cu2+ to facilitate the formation of S100A13 heterotetrameric complexes and these signal peptideless polypeptides have been implicated as regulators of vascular injury in vivo, we examined the ability of Cu2+ chelation to repress neointimal thickening in response to injury, Mandinov et al suggest that intracellular copper may be involved in mediating the response to injury in vivo by its ability to regulate the stress-induced release of IL-1alpha by using the nonclassical export mechanism employed by human peripheral blood mononuclear cells in vitro Copper chelation represses the vascular response to injury. Mandinov L, Mandinova A, Kyurkchiev S. Kyurkchiev D, Kehayov I, Kolev V, Soldi R, Bagala C, de Muinck E D, Lindner V, Post M J, Simons M, Bellum S, Prudovsky I, Maciag T Proc Natl Acad Sci USA. 2003; 100:6700-5).

Copper is involved in the promotion of angiogenic and inflammatory events in vivo and, although recent clinical data has demonstrated the potential of Cu2+ chelators for the treatment of cancer in man, the mechanism for this activity remains unknown. Mandinova et al in this context reported that IL-1alpha is a Cu2+-binding protein and human U937 cells, like NIH 3T3 cells, release IL-1alpha in response to temperature stress in a Cu2+ dependent manner. The authors conclude that because Cu2+ chelation also represses the release of FGF1, the ability of Cu2+ chelators to potentially serve as effective clinical anti-cancer agents may be related to their ability to limit the export of these proinflammatory and angiogenic signal peptideless polypeptides into the extracellular compartment. (Mandinova A, Soldi R, Graziani I, Bagala C, Bellum S, Landriscina M, Tarantini F, Prudovsky I, Maciag T. S100A13 mediates the copper-dependent stress-induced release of IL-1alpha from both human U937 and murine NIH 3T3 cells. J Cell Sci. 2003; 116:2687-96).

Enhanced cardiac generation of peroxynitrite contributes to septic cardiomyopathy. Since matrix metalloproteinases (MMPs) are activated in vitro by peroxynitrite, Lalu et al demonstrated, for the first time, that lipopolysaccharide induced cardiac dysfunction is associated with a loss in ventricular MMP-2 activity and the release of MMP-9 from the heart. MMP inhibitors can significantly preserve cardiac mechanical function during septic shock. (Lalu M M, Gao C Q, Schulz R. Matrix metalloproteinase inhibitors attenuate endotoxemia induced cardiac dysfunction: a potential role for MMP-9. Mol Cell Biochem. 2003; 251:61-6).

Sepsis precipitates a systemic inflammatory stimulus that causes systemic release of cytokines and sequestration of polymorphonuclear neutrophils, resulting in degranulation of matrix metalloproteinases (MMPs), which causes extracellular matrix basement membrane degradation. One of the important anti-inflammatory properties of tetracyclines is their ability to inhibit MMPs. The results of Maitra et al, indicate the beneficial effect of CMT-3 in preventing the increase in transaminases, NO, MMP-9, gelatinase activity, and the ensuing septic shock. (Maitra S R, Bhaduri S, Valane P D, Tervahartiala T, Sorsa T, Ramamurthy N. Inhibition of matrix metalloproteinases by chemically modified tetracyclines in sepsis. Shock. 2003; 20: 280-5).

Neutrophil activation with concomitant matrix metalloproteinase-2 (MMP-2) and matrix metalloproteinase-9 (MMP-9) release has been implicated in the development of sepsis-induced acute lung injury. Steinberg et al hypothesized that COL-3, a chemically modified tetracycline known to inhibit MMP-2 and MMP-9, would reduce lung injury and improve survival in rats following cecal ligation and puncture (CLP). Inhibition of MMP-2 and MMP-9 by COL-3 in a clinically relevant model of sepsis-induced acute lung injury reduces pulmonary injury and improves survival in a dose-dependent fashion. Steinberg et al results suggest that prophylactic treatment with COL-3 in high-risk patients may reduce the morbidity and mortality associated with sepsis-induced acute respiratory distress syndrome. (Steinberg J, Halter J, Schiller H J, Dasilva M, Landas S, Gatto L A, Maisi P, Sorsa T, Rajamaki M, Lee H M, Nieman G F. Metalloproteinase inhibition reduces lung injury and improves survival after cecal ligation and puncture in rats. J Surg Res. 2003; 111:185-95).

Endothelial adhesion molecule expression and monocyte recruitment are causal events in human atherosclerosis, and are believed to be caused, in part, by oxidative stress. Because redox-active transition metal ions, such as iron and copper, play an essential role in the generation of free radicals and the initiation and propagation of lipid peroxidation, we hypothesized that transition metal ions may also be involved in endothelial activation. Zhang and Frei investigated the effects of the intracellular iron-chelator, desferrioxamine (DFO), and the intracellular copper-chelator, neocuproine (NC), on TNFalpha-induced expression of adhesion molecules in human aortic endothelial cells (HAEC). The data of the two investigators suggest that intracellular, but not extracellular, transition metal ions mediate inflammatory cytokine-induced SP-1 activation and adhesion molecule expression in endothelial cells. (Zhang W J, Frei B. Intracellular metal ion chelators inhibit TNFalpha-induced SP-1 activation and adhesion molecule expression in human aortic endothelial cells. Free Radic Biol Med. 2003; 34:674-82).

Pulmonary complications from pancreatitis involve parenchymal destruction via proteolytic enzymes. Matrix metalloproteinases (MMPs) may play an important role in pulmonary injury following acute severe pancreatitis. Muhs et al hypothesized that local and distant organ injury would be decreased by the presence of an MMP inhibitor following severe acute pancreatitis Pancreatitis results in increased local and distant MMP activity. Pulmonary and pancreatic injury following acute pancreatitis can be abrogated by treatment with an MMP inhibitor which may result in decreased morbidity and mortality. (Muhs B E, Patel S, Yee H, Marcus S, Shamamian P. Inhibition of matrix metalloproteinases reduces local and distant organ injury following experimental acute pancreatitis. J Surg Res. 2003; 109:110-7).

Bacterial sepsis is characterized by a systemic inflammatory state, with activation of numerous cell types. Phagocytes participate in this phenomenon by secreting various proinflammatory cytokines and enzymes. Matrix metalloproteinases (MMPs) are produced by phagocytes and are thought to play an important role in processes of cell transmigration and tissue remodeling. Pugin et al show that endotoxin (lipopolysaccharide [LPS]) and other inflammatory mediators, such as tumor necrosis factor (TNF), interleukin-8, and granulocyte colony-stimulating factor, induce a rapid (within 20 min) release of gelatinase-B zymogen in whole human blood, as determined by gelatin zymography. The polymorphonuclear neutrophil was identified as the cell responsible for this rapid secretion, as a result of the release of preformed enzymes stored in granules. These data indicate that MMPs are released in whole blood in response to various inflammatory mediators and that they could serve as sensitive and early markers for cell activation during the course of bacterial sepsis. (Pugin J, Widmer M C, Kossodo S, Liang C M, Preas H L 2nd, Suffredini A F. Human neutrophils secrete gelatinase B in vitro and in vivo in response to endotoxin and proinflammatory mediators. Am J Respir Cell Mol Biol. 1999; 20:458-64).

Thrombin-activatable fibrinolysis inhibitor (TAFI) circulates as an inactive proenzyme of a carboxypeptidase B-like enzyme (TAFIa). It functions by removing C-terminal lysine residues from partially degraded fibrin that are important in tissue-type plasminogen activator mediated plasmin formation. TAFI was classified as a metallocarboxypeptidase, which contains a Zn(2+), since its amino acid sequence shows approximately 40% identity with pancreatic carboxypeptidases, the Zn(2+) pocket is conserved, and the Zn(2+) chelator o-phenanthroline inhibited TAFIa activity. Marx et al, showed that TAFI contained Zn(2+) in a 1:1 molar ratio. o-Phenanthroline inhibited TAFIa activity and increased the susceptibility of TAFI to trypsin digestion. TAFIa is spontaneously inactivated (TAFIai) by a temperature-dependent intrinsic mechanism. The lysine analogue epsilon-ACA, which stabilizes TAFIa, delayed the o-phenanthroline mediated inhibition of TAFIa. They investigated if inactivation of TAFIa involves the release of Zn(2+). However, the zinc ion was still incorporated in TAFIai, indicating that inactivation is not caused by Zn(2+) release. After TAFIa was converted to TAFIai, it was more susceptible to proteolytic degradation by thrombin, which cleaved TAFIai at Arg(302). Proteolysis may make the process of inactivation by a conformational change irreversible. Although epsilon-ACA stabilizes TAFIa, it was unable to reverse inactivation of TAFIa or R302Q-rTAFIa, in which Arg(302) was changed into a glutamine residue and could therefore not be inactivated by proteolysis, suggesting that conversion to TAFIai is irreversible. (Marx P F, Bouma B N, Meijers J C. Role of zinc ions in activation and inactivation of thrombin-activatable fibrinolysis inhibitor. Biochemistry. 2002; 41:1211-6).

Endogenous copper can play an important role in postischemic reperfusion injury, a condition associated with endothelial cell activation and increased interleukin 8 (IL-8) production. Excessive endothelial IL-8 secreted during trauma, major surgery, and sepsis may contribute to the development of systemic inflammatory response syndrome (SIRS), adult respiratory distress syndrome (ARDS), and multiple organ failure (MOF). No previous reports have indicated that copper has a direct role in stimulating human endothelial IL-8 secretion. The results of Bar-Or, suggest that Cu(II) may induce endothelial IL-8 by a mechanism independent of known Cu(I) generation of reactive oxygen species. Furthermore, in vivo studies are warranted to determine if copper is involved in the pathogenesis of systemic inflammation and if Cu(II) chelation can reduce this IL-8-induced endothelial inflammatory response. (Bar-Or D, Thomas G W, Yukl R L, Rael L T, Shimonkevitz R P, Curtis C G, Winkler J V. Copper stimulates the synthesis and release of interleukin-8 in human endothelial cells: a possible early role in systemic inflammatory responses. Shock. 2003; 20:154-8).

Phenanthroline is a zinc-chelator that inhibits biological activities of matrix metalloproteinases (MMPs). Over-expression of MMPs can accelerate tissue destruction and disrupt subsequent tissue repair. The effects of phenanthroline in two rat models of inflammatory bowel disease) are evaluated. Medina et al have found that in the transmural colitis induced by trinitrobenzensulphonic acid model, phenanthroline treatment significantly reduced colonic strictures; in the distal colitis caused by dextran sulphate sodium model, phenanthroline significantly decreased scores of epithelial injury. The authors concluded that although phenanthroline did not modify the activity of inflammatory mediators, this compound substantially reduced intestinal injury associated with tissue remodeling. It is noted that phanquinone is a phenanthroline (4,7-phenanthroline-5,6-quinone). (Medina C, Videla S, Radomski A, Radomski M, Antolin M, Guarner F, Vilaseca J, Salas A, Malagelada Therapeutic effect of phenanthroline in two rat models of inflammatory bowel disease. Scand J Gastroenterol. 2001; 36:1314-9).

Florianczyk describes that metallothioneins that are intracellular proteins whose biological function is zinc or copper regulation as well as detoxification of toxic metals and have another function to sweep away free radicals. MT synthesis induction is stimulated by such factors as metallic ions, free radicals, cytokines, lymphokines and stress. An increased intracellular metallothionein expression was found in many human and animal neoplasms. Copper functions as cofactor in various redox enzymes. At the same time, copper is very toxic to both eukaryotic and prokaryotic cells. Copper ions can bind to proteins and nucleic acids and cause the oxidation of lipids and proteins. The formation of deleterious free radicals is also enhanced by copper ions. For cell viability, regulation of intracellular Copper activity is thus crucially important and mechanisms must exist for the homeostasis of copper. An elevated copper level is noted in many types of neoplastic tissue. (Florianczyk B. Copper and metallothioneins in cancer cells Ann Univ Mariae Curie Sklodowska 2003; 58.390-3).

The MMPs play a key role in the normal physiology of connective tissue Brew et al. An important mechanism for the regulation of the activity of MMPs is via binding to a family of homologous proteins (TIMP-1 to TIMP-4). The two-domain TIMPs are of relatively small size, yet have been found to exhibit several biochemical and physiological/biological functions, including inhibition of active MMPs, proMMP activation, cell growth promotion, matrix binding, inhibition of angiogenesis and the induction of apoptosis. Mutations in TIMP-3 are the cause of Sorsby's fundus dystrophy in humans, a disease that results in early onset macular degeneration. Recently the highresolution structures of TIMPs have been elucidated and their complexes with metalloproteinases, and the results of mutational and other studies of structure-function relationships that have enhanced our understanding of the mechanism and specificity of the inhibition of MMPs by TIMPs. Several intriguing questions, such as the basis of the multiple biological functions of TIMPs, the kinetics of TIMP-MMP interactions and the differences in binding in some TIMP-metalloproteinase pairs are discussed which, though not fully resolved, serve to illustrate the kind of issues that are important for a full understanding of the interactions between families of molecules. (Brew K, Dinakarpandian D, Nagase H. Tissue inhibitors of metalloproteinases: evolution, structure and function. Biochim Biophys Acta. 2000; 1477:267-83).

Plantner et al have shown that MMPs have increasingly been shown to be associated with diseases involving neovascularization and/or abnormal cellular migration or proliferation. A number of diseases of this type affect the retina. MMP-2, the most abundant MMP interphoto receptor matrix and vitreous, was measured with respect to age in normal human donor eyes and compared to donors with age-related macular degeneration. The level of MMP-2, was nearly doubled specifically in retinal pigment epithelium-associated interphoto receptor matrix from eyes with age-related macular degeneration, suggesting that MMP-2 may be associated with the changes that occur in age-related macular degeneration, especially the neovascularisation which accompanies the exudative form of the disease. (Plantner J J, Jiang C and 5 mine A Increase in interphotoreceptor matrix gelatinase A (MMP-2) associated with age-related macular degeneration, Exp Eye Res 1998; 67:637-45)

Kodonosono et al showed that MMPs are associated with neovascularisation and in particular MMP-7 was expressed in Bruch membrane of choroidal neovascular membranes in age-related macular degeneration. MMP-7 may be an important factor for the development of the sub-macular neovascular membrane in age-related macular degeneration, (Kadonosono K, Yazama F, Itoh N, Sawada H, Ohno S Expression of matrix metalloproteinase-7 in choroidal neovascular membranes in age-related macular degeneration. Am J Opthalmol. 1999; 128:382-4).

Further Jomary et al, found that the localisation of MMP-3 in neurodegenerative retinal disease is implicated in the regulation of remodelling of the ECM. The level of mRNA coding for TIMP-3 is increased in retinas affected by the photoreceptor degenerative disease, simplex retinitis pigmentosa, and mutations in TIMP-3 are associated with an inherited form of macular dystrophy. The comparison of TIMP-3 protein expression in normal retina and in those affected by R P and by age-related macular degeneration. Immunoreactive TIMP-3 is present in normal retinal pigment epithelium, and in degenerative retinas particularly at Bruch's membrane and additionally in photoreceptor-retaining regions in simplex R P. The pattern suggests a role for TIMP-3 in normal retinal homeostasis, and, in the disease state, in the modulation of extracellular matrix metabolism and neovascularisation. (Jomary C, Neal M J, Iwata K, Jones S E. Localization of tissue inhibitor of metalloproteinases-3 in neurodegenerative retinal disease. Neuroreport 1997; 8:2169-72).

Matrix metalloproteinases and metalloproteinase inhibitors are present in human interphoto receptor matrix and vitreous and it was established that MMPs and TIMPs were present in human interphoto receptor matrix, and vitreous. It was demonstrated that it is most likely that MMPs and TIMPs are involved in normal turnover within the ECM that surround the neural retina and play a role in a number of retinal diseases, particularly proliferative diabetic retinopathy and age-related macular degeneration.

More particularly in Sorsby's fundus dystrophy (Felbor U, Stohr H, Amann T, Schonherr U, Apfelstedt-Sylla E, Weber B H. A second independent Tyr168Cys mutation in the tissue inhibitor of metalloproteinases-3 (TIMP3) in Sorsby's fundus dystrophy. 3 Med. Genet. 1996; 33:233-6) showed that mutation occurrence of the Tyr168Cys mutation in an Sorsby's fundus dystrophy patient seem to affect the C-terminal region of the mature TIMP3 protein. In addition, all known mutations cause a change of an amino acid to a cysteine residue. This suggests a critical role for the additional C-terminal free thiol group in Sorsby's fundus dystrophy pathogenesis.

The evaluation of the gene encoding the tissue inhibitor of metalloproteinases-3 in various maculopathies (Felbor U, Doepner D, Schneider U, Zrenner E, Weber B H. Evaluation of the gene encoding the tissue inhibitor of metalloproteinases-3 in various maculopathies. Invest Opthalmol V is Sci. 1997; 33:1054-9) showed that the mutations in the gene encoding the tissue inhibitor of TIMP3 cause Sorsby's fundus dystrophy, characterised by ECM irregularities in Bruch's membrane. In the 217 patients analysed Felbor et al, identified one sequence alteration (a G-to-C base change) in the 5′-untranslated region in a patient with age related macular degeneration. It is suggested that TIMP-3 is not a major factor in the cause of age related macular degeneration, adult vitelliform macular dystrophy, central areolar choroidal dystrophy, syndrome-associated macular dystrophies, cone-rod dystrophy, and in a group with unspecified macular degeneration and that only Sorsby's fundus dystrophy appears to be only associated with mutations in TIMP3. Vettakkorumakankav and Ananthanarayanan (Ca(2+) and Zn(2+) binding properties of peptide substrates of vertebrate collagenase, MMP-1. Biochim Biophys Acta. 1999; 1432:356-70) showed that TIMP binds only on zinc with definite stoichiometries.

De La Paz et al showed that MMPS and their endogenous TIMPs are present in human vitreous and may be involved in the pathogenesis of vitreo-retinal diseases. (De La Paz M A, Itoh Y, Toth C A, Nagase H. Matrix metalloproteinases and their inhibitors in human vitreous. Invest Opthalmol V is Sci. 1998 Jun; 39(7):1256-60).

Recently synthetic, potent, low molecular weight MMP inhibitors have been developed and, over the past five years and that these agents have begun clinical testing in patients with cancer, rheumatoid arthritis, osteoarthritis and acute macular degeneration.

Copper has a dual role in cancer by increasing angiogenesis and promoting inflammation. Copper is incorporated in the extracellular matrix and at the same time and on the same tissue levels copper interacts with epidermal growth factor, fibroblast growth factor, granulocyte platelet derived growth factor, colony stimulating factor, tumor necrosis factor alpha, vascular endothelial growth factor, zinc/copper superoxidase dismutase, cathepsin, gelatinases, stromelysin, urokinase-type plasminogen activator, zinc dependent proteases and endopeptidases, interleukin-1, interleukin-6, interleukin-8, nitric oxide synthetase and phospholipase.

The role of copper in cancer promotion through inflammation and angiogenesis is now well understood. Copper is incorporated in the extracellular matrix that forms the very structure of blood vessels. Without it, they can not function, and growth of new blood vessels stops. In other words, copper-reduction blocks angiogenesis by “switching” the endothelial cell into the apoptosis (programmed cell death) pathway, or quiescence, and the cancer remains dormant.

For numerous malignancies (including lymphomas and leukemias) tumor incidence, progression, severity and relapse are all associated with high levels of copper in serum. Numerous studies since the 1970s established excess copper levels and high copper/zinc ratios as prognostic indicators for lymphoma where higher levels correspond to more aggressive disease. Now that it is known that high copper levels promote angiogenesis, perhaps this can help explain the prognostic effect of high copper levels. A 1988 study in Shanghai simultaneously determined the Cu and Zn of 173 lymphoma patients by atomic absorption spectrophotometry. The study concluded that copper may be used as prognostic indicators for monitoring disease activity and response to therapy in malignant lymphoma.

Ceruloplasmin (Cp) is a glucoprotein that transports copper and was found to be significantly elevated in advanced stages of solid malignant tumors and increases up to four- to eight-fold during malignant progression. Data analysis in another study suggested Cp as a good diagnostic marker of cancer. Often before tumors become palpable, tumor regression returns Cp levels to normal. From this evidence it appears clear that tumors of all types have at their disposal the means to increase copper and Cp levels for purposes of angiogenesis.

The involvement of inhibitors of zinc dependent proteinases such as the MMPs in cancer has been the subject of continuous scientific interest for at least years and investigations have pointed 3 not only to a role of inhibitors of MMPs in invasion and metastasis but also in tumour growth, apoptosis, transformation, and angiogenesis. The inhibitors of MMPs cannot only block tumor invasion and metastasis but also inhibit the growth of primary tumors. As an example, leukemia cells secrete in tissue culture MMPs, one of which is the known MMP-9. It has been shown that chemical chelators, such as EDTA and phenanthrolene, are able to inhibit the activity of said MMPs and halt the degradation of the matrix constituents (Dittmann K H, Lottspeich F, Ries C, Petrides P E Leukemic cells (HL-60) produce a novel extracellular matrix-degrading proteinase that is not inhibited by tissue inhibitors of matrix metalloproteinases. Exp Hematol. 1995; 23:155-60).

Clioquinol (5-chloro-7-iodo-8-hydroxyquinoline) was previously frequently used for the treatment of various disorders, such as amoebiasis and non-specific infectious diarrhea. However, the use of clioquinol was stopped due to the presumption that clioquinol caused subacute myelo-opticoneuropathy (SMON).

Renewed interest has been evinced in clioquinol recently as it has been shown to be effective in the treatment of Helicobacter pylori (WO 9/31199) and neurotoxic injury (WO 97/09976). Furthermore, in USTPO 980,914, clioquinol has been suggested for the treatment of Parkinson's disease and in WO 98/06403, clioquinol has been suggested for the treatment of Alzheimer's disease. In WO 99/34807 it is stated that the hydrophobic binding of vitamin B₁₂ to a metabolite of clioquinol (clioquinol glucuronide) is believed to cause the vitamin B₁₂ to be excreted from the body together with clioquinol glucuronide, thus preventing resorption of vitamin B₁₂, which would eventually lead to a vitamin B₁₂ deficiency. Therefore, vitamin B₁₂ deficiency is believed to be, at least to some extent, the underlying cause of SMON.

Phanquinone (4,7-phenanthroline-5,6-dione) has hitherto been used for the treatment of various disorders, such as amoebiasis. Phanquinone has been sold by CIBA-GEIGY under the trademark ENTOBEX. In contrast to clioquinol no adverse side effects have been detected when phanquinone is used in the normal dosage range.

In the past, an antiamoebic pharmaceutical preparation containing both clioquinol and phanquinone has been sold by CIBA GEIGY under the trademark Mexaforme. However, the marketing of this preparation was stopped when it was supposed that clioquinol caused SMON.

Also phanquinone has received renewed interest in recent years and has been suggested for the treatment of Alzheimer's disease in WO 99/09981 and its effect on the beta-amyloid described.

### SHORT DESCRIPTION OF THE INVENTION

The invention relates to the use of chelators in cancer, inflammatory diseases, rheumatic acute coronary and macular disease aiming to a combined effect on two pivotal metalions, zinc and copper as well as zinc or copper dependent metalloproteins that are pathologically increased or imbalanced or deregulated or disturbed.

Thus in one aspect of the invention relates to the use of at least one copper and/or zinc specific chelator(s) for the treatment of cancer, inflammatory, immune acute coronary and macular disease.

It has surprisingly been found that the cancer, inflammatory diseases, rheumatic acute coronary and macular disease can efficiently be treated according to the invention leading to a cessation of the development of the diseases or even a reduction of the malignancy depending on the particular disease and developmental stage thereof.

Further the inventor has realized that the combination of at least one copper specific chelator and at least one zinc specific chelator has a more potent and broad effect on said indications than should have been expected based on the knowledge of the effect of each of the particular copper and/or zinc specific chelator.

Thus in a preferred aspect the invention relates to the use of a combination of at least one copper specific chelator and at least one zinc specific chelator for the treatment of cancer, inflammatory diseases, rheumatic acute coronary and macular disease.

Clioquinol is a preferred zinc specific chelator and phanquinone is a preferred copper specific chelator according to the invention.

Pharmaceutical compositions, kits etc., comprising at least one copper and/or zinc specific chelator form other aspects of the invention.

### DETAILED DESCRIPTION OF THE INVENTION

The copper and/or zinc specific chelators according to the invention may in principle be any such chelator, however it is preferred that the chelator have suitable properties to be used as a pharmaceutical compounds, such as a suitable clearence rate, low toxicity, preferably able to be assimilated from the gastro-intestinal tract etc.

It is well known that chelators often have the ability to bind several metal ions often with varying binding strengths. According to the invention the a chelator having specificity for copper may be selected among chelators having a greater affinity to copper ions than to other metal ions, and preferably is the binding constant higher than 5.0, preferably higher than 6.0 and most preferably higher than 7.0. A chelator having specificity for zinc may be selected among chelators having a greater affinity to copper ions than to other metal ions, and preferably is the binding constant higher than 5.0, preferably higher than 6.0 and most preferably higher than 7.0.

The skilled person will appreciate that binding constants for various chelators may be found in the chemical literature or may be determined using generally known well established procedures. It is therefore within the skills of the average practitioner to select suitable chelators for the present invention.

Examples of chelators for use according to the invention include: clioquinol, phanquinone, 8-hydroxyquinoline, 5,7-dichloro-8-hydroxyquinoline, 5,7-dibromo-8-hydroxyquinoline, 2-methyl-8-hydroxyquinoline or 8-hydroxyquinaldine, 5,7-dichloro-2-methyl-8-hydroxyquinoline, 5,7-dichloro-8-hydroxy quinaldine, 5-methyl-oxine (5-methyl-8-hydroxyquinoline), 2-mercaptopyridine-N-oxide, 5-fomyl-, 5-iodo-, 5-fluoro-, 5-acetyl-, and 5-methoxymethyl-8-hydroxyquinoline, ethyl 5-(8-hydroxyquinolyl)acetate, methyl-5(8-hydroxyquinolyl)acetate, ethyl 5-(8-hydroxyquinolyl)acetate, 2,7,8,-trihydroxyquinoline, indole-3-acetaldehyde to 4-hydroxyquinoline, 4-hydroxyquinoline-3-carboxylates, 5-phenylethyl-4-hydroxyquinoline-3-carboxylic acids, dibromo-8-bezoil-oxyquinoldine, arylglyoxal N-7-amino-5-substituted 8-hydroxyquinoline hemiacetals, and 5-phenylglyoxylidenamin-8-hydroxyquinolines, alpha, alpha′-dipyridyl, 8-hydroxyquinoline, 2,2′,2″-tripyridine, 2,6-dihydroxyquinoline, 5-formyl-1-methoxycarbonyl-4,6,8-trihydroxyphenazine, 5- or 7-methylthio-8-hydroxyquinoline derivatives, 4-hydroxyquinoline-2 and -3-carboxylic acids, 5-phenylethyl-4-hydroxyquinoline-3-carboxylic acids, 2-n-alkyl-4-hydroxyquinoline derivates, O-acetyl-8-hydroxyquinoline, S-acetyl-8-mercaptoquinoline, 5,7 dibromo-8-bezoil-oxyquinoldine, 5-phenylglyoxylidenamin-8-hydroxyquinolines, ethyl 6,7-di-isobutoxy-4-hydroxyquinoline-3-carboxylate, 5-chloro-8-hydroxyquinoline, 5-chloro-8-hydroxyquinoline (chloroxine) esters of carboxylic acid, m-phenylenediamine, 1aH-oxazirino[2,3-a]quinoline la-carbonitrile and its sub-stituted derivatives to the corresponding 3-hydroxyquinoline derivatives, 6,7-dimethoxy-4-hydroxyquinoline hydrochloride, 4-nitroquinoline 1-oxide, nitroxoline, 8-hydroxy-quinoline-7-carboxylic acid, hydroxyquinoline sulfate, chloro-hydroxy-quinoline, 8-hydroxy-quinoline-5-sulfonic acid, derivatives of 1,2,3,4-tetrahydro-quinoline, 2-heptyl-4-hydroxyquinoline-N-oxide or 2-nheptyl-4-hydroxyquinoline-N-oxide, 4-hydroxy quinoline-3-carboxylic acids (7 substituted benzyl-oxy, phenethyl-oxy or phenoxy-ethoxy), 6-hydroxyquinoline, 6-nitro quinoline, 8-nitroquinoline, 6-methyl quinoline, 8-methyl quinoline, 8-hydroxyquinoline, 5,7-dibromo-quinoline, 2-n-nonyl-4-hydroxy[3-3H]quinoline, 2n-nonyl-4-hydroxyquinoline-N-oxide 8-hydroxyquinoline-5-sulfonic acids, 2,2′,2″-terpyridine complexes, 8-hydroxyquinoline 5-sulfonic acid, 5-nitro-8-hydroxyquinoline, 2-mercaptoquinoline-N-oxide, 5,6-benzoquinoline, 5-methyl-8-hydroxyquinoline 4-hydroxyquinoline, 2-n-alkyl-4-hydroxyquinoline, ethylenediaminetetraacetic acid, O-phenanthroline, 1,10-phenanthroline, 2,9-dimethyl-1,10-phenanthroline, (2′-hydroxyphenyl)pyridine, rhodotorulic acid, mycobactin P, 8-hydroxyquinoline-7-carboxylate, 5-substituted 8-hydroxyquinolines, 5-chlor-8-hydroxyquinoline, 4-hydroxy quinoline, 6-, 7-, or 8-substituted-4-hydroxyquinoline-3-carboxylic acids, 5-7 dibromo-8-hydroxyquinoline, 2,2′-bipyridine, 8-hydroxy quinoline esters, halogen derivatives of salicylanilide, batimastat, hydroxamic acids and fenamates.

As examples of chelators having a high specificity for copper can be mentioned: phanquinone (4,7-phenanthroline-5,6-quinone), ethylenediaminetetraacetic acid. O-phenanthroline, 1,10-phenanthroline and 2,9-dimethyl-1,10-phenanthroline where phanquinone is preferred.

As examples of chelators having a high specificity for zinc can be mentioned: clioquinol, 8-hydroxy-quinoline, 5,7-di-iodo-8-hydroxy-quinoline and 5,7-dichloro-8-hydroxyquinoline, where clioquinol is preferred.

In one preferred embodiment the invention relates to the use of a combination of at least one copper specific chelator and at least one zinc specific chelator for the treatment of cancer, inflammatory, immune acute coronary and macular disease.

Preferably the copper specific chelator is phanquinone and the zinc specific chelator is clioquinol.

The one or more copper specific chelator(s) may be administered to the patient in need of the treatment according to the invention simultaneously or sequentially to the administration of the one or more zinc specific chelator(s). It is preferred to administer the one or more copper specific chelator(s) and the one or more zinc specific chelator(s) with a timing so that a therapeutic concentration of the one or more copper specific chelator(s) and a therapeutic concentration of the one or more zinc specific chelator(s) is reached at least in part of the period of the treatment.

The one or more copper specific chelator(s) and/or the one or more zinc specific chelator(s) is (are) according to the invention administered to a patient in need therefore in form of one or more pharmaceutical composition(s). The pharmaceutical compositions according to the invention may be prepared according to well known procedures for formulating pharmaceutical compositions as it will be well known within the area.

The pharmaceutical compositions according to the invention is explained in further details below with reference to clioquinol and phanquinone even thought the skilled person will appreciate that the teaching applies likewise to other chelators according to the invention.

The pharmaceutical composition manufactured using clioquinol preferably comprises one or more pharmaceutical acceptable carriers and, optionally, one or more further active constituent(s). The carrier(s) must be “acceptable” in the sense of being compatible with the other ingredients of the composition and not deleterious to the recipients thereof. In a preferred embodiment, the clioquinol and, optionally, further active constituents in the pharmaceutical composition are purified.

It will be appreciated that the amount of clioquinol and, optionally, further active constituents required for said treatment or prevention will vary according to the route of administration, the disorder to be treated, the condition, age, the file history of the subject, and the galenic formulation of the pharmaceutical composition, etc. When treating a patient diagnosed as having a pathological condition influenced by the action of at least one of the zinc or copper dependent proteins, the amount of clioquinol or phanquinone is preferably effective to provide for at least a partially modulation or inhibition of one of them.

In general, a suitable therapeutically effective amount of clioquinol in the pharmaceutical composition for oral use is, for example, 100 mg to 1 g, to more preferred 250 mg to 500 mg. The amounts of phanquinone for oral use, is for example 5 mg and 500 mg, to more preferred 50 mg to 100 mg.

The combination of clioquinol and phanquinone for a suitable therapeutically effective amount in the pharmaceutical composition for oral use is, for example, 100 mg to 1 g, and more preferred 250 mg to 500 mg. of clioquinol and 5 mg to 500 mg and more preferred 50 mg to 100 mg of phanquinone.

The most suitable formulation that will modulate simultaneously on the target zinc and copper dependent proteins that are deregulated or pathologically increased, is a combination of both clioquinol and phanquinone. The daily effective amount for administration in the pharmaceutical composition, comprises 100 mg to 6 g clioquinol and 5 mg to 2 g phanquinone. The administration, for example, can be at high dosages for periods of one week to three months and at low dosages for periods from three months to lifetime. The high dosage administration can be preferably parenteral and the low dosage oral. The combination therapy with the two chelating agents will obtain a stronger action on the modulation of the target proteins and will broaden the spectrum of the chelating effect.

In general, a suitable therapeutically effective amount of clioquinol in the pharmaceutical composition is, for example, 100 mg to 6 g, preferably 250 mg to 1 g. The amounts of phanquinone, 8-hydroxy-quinoline, and 5,7-dichloro-8-hydroxyquinolineare preferably 5 mg and 1 g mg to more preferred 50 mg to 100 mg. The amounts of 5,7-di-iodo-8-hydroxy-quinoline are preferably from 50 mg to 5 g and more preferred 500 mg to 1 g. If the pharmaceutical composition in addition to the chelators mentioned comprises further active constituents they may be in the same composition for administering in combination concurrently, or in different compositions for administering substantially simultaneously but separately, or sequentially. If the active constituents are administered sequentially, the further active ingredients may be administered prior or subsequently to the administering of clioquinol.

Pharmaceutical formulations include those suitable for parenteral and oral use. The parenteral formulations can be for intramuscular, intravenous and intracoronary administration.

The preferred dosage for clioquinol is 100 mg to 6 g, for phanquinone is 5 mg to 2 g, for the combination of clioquinol and phanquinone is 100 mg to 4 g and 5 mg to 2 g respectively.

Pharmaceutical formulations include those suitable for parenteral, oral, transdermal, ophthalmic or suppository route. The parenteral formulations can be given by intramuscular, intra venous, intracoronary or intrathecal administration. The preferred dosage for clioquinol is 100 mg to 6 g of 1 percent or hydroxyethylcellulose or carboxymethylcellulose of calcium or sodium salts or other suitable dispersants and solubilising agents such as croscarmellose sodium, crospovidone, povidone, sodium alginate, magnesium aluminium silicate, cyclodextrin and colloidal silicon dioxide or polyethylene glycol. For phanquinone is of 5 mg to 2 g, for the combination of clioquinol and phanquinone is 100 mg to 5 g and 5 mg to 2 g respectively.

Pharmaceutical formulations include those suitable for parenteral, oral, transdermal or suppository route clioquinol or 5-Chloro-7-iodo-8-hydroxyquinoline, phanquinone or 4,7-phenanthroline-5,6-quinone, as well as 8-hydroxy-quinoline, 5,7-di-iodo-8-hydroxyquinoline and 5,7-dichloro-8-hydroxyquinoline is from 5 mg to 6 g.

More preferred is for clioquinol or 5-chloro-7-iodo-8-hydroxy-quinoline as well as for 8-hydroxy-quinoline, 5,7-dichloro-8-hydroxyquinoline and 5,7-di-iodo-8-hydroxyquinoline 500 mg three times daily orally and 1.5 g daily parenterally, for phanquinone or 4,7-phenanthroline-5,6-quinone 50 mg three times daily orally and 150 mg daily parenterally.

More preferred by suppository, is for clioquinol, 8-hydroxy-quinoline, 5,7-di-iodo-8-hydroxyquinoline and 5,7-dichloro-8-hydroxyquinoline 500 mg three times daily and for phanquinone 50 mg three times.

More preferred by transdermal route is a cream of 3% for clioquinol and 3% for phanquinone or a cream containing each of clioquinol and phanquinone at a concentration of 3%. For 8-hydroxy-quinoline, 5,7-dichloro-8-hydroxyquinoline and 5,7-di-iodo-8-hydroxyquinoline is a cream of 3% for a three time daily application.

For eye application the preferred opthalmologic preparation is an ointment of clioquinol and phanquinone at a concentration of 1 to 2% of each constituent.

The preferred route for high-dosage chronic use is the oral. For high dosage short period treatments the preferred route is the parenteral. For acute coronary syndromes such unstable angina and acute myocardial infract the preferred route is parenteral as well as intracoronary for invasive cardiology treatments such as angioplasty or coronary reperfusion.

An example of clioquinol and phanquinone might be in the intensive care severe sepsis patient treatment. A combination of clioquinol 6 g daily and phanquinone 2 g daily by intravenous infusion for 120 hours might be added to the standard procedures as defined in the Guidelines for sepsis. Another example in the intensive care sepsis treatment may be the addition of clioquinol 2 g daily by intramuscular injection added to the standard procedures as defined in the Guidelines for sepsis. Another suitable patient in intensive care might be a patient with acute pancreatitis. Such a patient might be administered the same dosages as in severe sepsis however for a length of treatment period of seven days followed by oral administration of clioquinol 500 mg three times daily for another four weeks. Mineral and multiple vitamin supplements will be added accordingly.

An example is the use of clioquinol and phanquinone might be in the acute setting of a catheterisation laboratory of invasive cardiology. When a patient suffering from acute myocardial infarct that is urgently admitted for reperfusion as an adjunct to the regular treatment and during the percutaneous coronary intervention clioquinol at a dosage of 4 g plus phanquinone in a dosage of 500 mg might be administered by intravenous infusion lasting 60 minutes. Alternatively clioquinol at a dosage of 1 g plus phanquinone in a dosage of 100 mg can be administered by intracoronary administration lasting 10 minutes. The treatment may be followed by oral administration of 250 mg of clioquinol plus 50 mg of phanquinone or 500 mg cioquinol alone, three times daily for one week. The aim of the clioquinol and phanquinone treatment is to stop the extracellular matrix destruction and protect the endothelial integrity of the culprit atheromatous plaque, reducing the probability or the consequences of a rupture and helping in the restoration of the endothelial and sub endothelial histology. Mineral and multiple vitamin supplements will be added accordingly.

Another example in the treatment in the acute setting of catheterisation laboratory for a patient suffering from non-ST-segment myocardial infarct (refractory unstable angina) that is urgently admitted for reperfusion. Clioquinol may be administered orally at a dosage of 2 g prior to the invasive procedure as an adjunct to the standard regular treatment. Following the reperfusion clioquinol at a dosage of 250 mg daily may be administered orally for one month.

Another different patient is suffering from a chronic stable angina with one episode or unstable angina non ST-segment elevation. The patient after hospitalization and appropriate medical treatment may be prescribed in addition to the standard therapy, 250 mg orally administered clioquinol twice daily for life-time. Mineral and multiple vitamin supplements will be added accordingly.

A different patient suffering from stable angina is treated in adjunct with the regular treatment with 250 mg of clioquinol alone or plus 50 mg of phanquinone three times daily for one year at least. The aim of the clioquinol and phanquinone treatment is to stabilise the atherosclerotic plaque and maintain the endothelial integrity of the cap by inhibiting the enzymatic destruction of the extracellular matrix and reducing the local inflammatory and immune response. Mineral and multiple vitamin supplements will be added accordingly.

A different patient suffering from rheumatoid arthritis in adjunct to the non steroidal anti-inflammatory agents and corticosteroids and other treatment, may be treated with 250 mg of clioquinol or alternatively with 25 mg of phanquinone three times daily for life-time. This patient will be getting mineral and multiple vitamin supplements for life-time. This treatment can be combined with 8-hydroxy-quinoline, 5,7-dichloro-8-hydroxy-quinoline and 5,7-di-iodo-8-hydroxyquinoline at a dosage of 250 mg three times daily for one month treatment during the acute exacerbations of the disease. The aim of the clioquinol and phanquinone treatment is to reduce the auto-immune process of self-destruction by reducing the inflammation and the neoangiogenesis and preserving the extracellular matrix tissue histological architecture. Mineral and multiple vitamin supplements will be added accordingly.

A different patient is suffering from diabetes with renal and neurological complications. This patient might be prescribed in addition to the regular antidiabetic and other appropriate therapies, 250 mg clioquinol three times daily for lifetime. To this treatment phanquinone or 4,7-phenanthroline-5,6-quinone, as well as 8-hydroxy-quinoline, 5,7-di-iodo-8-hydroxyquinoline and 5,7-dichloro-8-hydroxyquinoline 25 mg three times daily is added for seven days at monthly wash-out intervals. The aim of the quinolines or phanquinone treatment is to reduce the extracellular matrix architecture and to inhibit neoangiogenesis. This patient will be getting mineral and multiple vitamin supplements for life-time.

A patient suffering from ulcerative colitis may be treated in adjunction to sulfasalazine and adrenal corticosteroids with a combination of clioquinol and phanquinone to proven recurrences. The combination consisting of 250 mg of clioquinol alone or in combination to 25 mg of phanquinone administered three times daily. Mineral and multiple vitamin supplements will be added accordingly.

Another example refers to a patient is suffering from colorectal cancer with lung metastasis. This patient, further to the standard surgical and radiotherapy protocols, may be treated with monthly seven-day acute regimen of 4 g clioquinol plus 1 g phanquinone intravenous infusions lasting each two hours. The aim of the treatment is to reduce the neoangiogenesis and the metastatic lesions. The parenteral regimen is completed with a three times daily per os 250 mg clioquinol administration during the periods when no parenteral administrations are administered. This patient will be getting mineral and multiple vitamin supplements for life-time.

A patient is suffering from colorectal cancer with liver metastasis. This patient, further to the standard surgical and radiotherapy protocols, may be treated with monthly seven-day acute regimen of 2 g clioquinol plus 500 mg phanquinone intraveneous infusions lasting each two hours. The aim of the treatment is to reduce the neoangiogenesis and the metastatic lesions. The parenteral regimen may be completed with a three times daily per os 250 mg clioquinol administration during the periods when no parenteral administrations are administered. Mineral and multiple vitamin supplements will be added accordingly.

Another example is a patient with age related macular degeneration. Such a patient in addition to the regular therapy may be prescribed with a treatment of an opthalmologic ointment containing 1% of clioquinol or of an ointment of 0.1% of phanquinone for use before sleep and twice daily administration of clioquinol 2% eye drops for day use. The aim of the treatment is to reduce the neovascularisation and modulate the extracellular matrix metabolism. Mineral and multiple vitamin supplements will be added accordingly.

Another patient is suffering from advanced brain tumor with metastatic lesions in the spinal cord. This patient may be prescribed 5 g of clioquinol administered by infusion for seven days. To this treatment intrathecal administration of 1 g of clioquinol plus 500 mg of phanquinone may be administered once a day for three consecutive days. The patient will be managed in addition to the clioquinol treatment with the standard protocols of radiotherapy. The aim of the treatment is to reduce the neoangiogenesis and the metastatic lesions. Mineral and multiple vitamin supplements will be added accordingly.

A different patient with a stage III lung cancer with a tumor that has invaded the chest wall, and the nearest lymph nodes having had a wedge resection and receiving hyperfractionated radiation therapy, may be treated with clioquinol 6 g daily alone or in combination with phanquinone 2 g daily, by intravenous infusions for one week. The clioquinol and phanquinone treatment is given in adjunction to a paclitaxel and carboplatin standard protocol. The treatment is repeated every two months for a period of one year. Outside the periods with the intravenous high dosages the patient is administered 750 mg of clioquinol total daily dose or 50 mg of phanquinone total daily dose, both in three divided doses. This patient will be getting mineral and multiple vitamin supplements.

Thus, the pharmaceutical composition may be formulated as vials, ampoules, bottles, tablets, pills, syrups, capsules, suppositories, formulations for transdermal application, powders, especially lyophilized powders for reconstitution with a carrier for intravenous administration, etc. The pharmaceutical compositions are prepared using conventional carriers.

The term “carrier” refers to a diluent, adjuvant, excipient, or vehicle with which the therapy is administered. The carriers in the pharmaceutical composition may comprise a binder, such as microcrystalline cellulose, polyvinylpyrrolidone (polyvidone or povidone), gum tragacanth, gelatine, starch, lactose or lactose monohydrate is a disintegrating agent, such as alginic acid, maize starch and the like, a lubricant or may be prepared using surfactant, such as magnesium stearate, or sodium lauryl sulphate; a glidant, such as colloidal silicon dioxide; a sweetening agent, such as sucrose or saccharin; and/or a flavouring agent, such as peppermint, methyl salicylate, or orange flavouring.

Pharmaceutical formulations suitable for oral administration, e.g. tablets and pills, may be obtained by compression or molding, optionally with one or more accessory ingredients. Compressed tablets may be prepared by mixing the constituent(s), and compressing the mixture obtained in a suitable apparatus into tablets having a suitable size. Prior to the mixing, the clioquinol may be mixed with a binder, a lubricant, an inert diluent and/or a disintegrating agent and the further optionally present constituents may be mixed with a diluent, a lubricant and/or a surfactant.

In a preferred embodiment, free-flowing clioquinol or phanquinone powder is mixed with a binder, such as microcrystalline cellulose, and a surfactant, such as sodium lauryl sulphate, until a homogeneous mixture is obtained. Subsequently, another binder, such as polyvidone, is transferred to the mixture under stirring. Said mixture is passed through granulating sieves and dried by desiccation before being compressed into tablets in a standard compressing apparatus.

In a second preferred embodiment, free-flowing clioquinol powder is mixed with surfactants and/or emulsifying agents, such as Sapamine (N-(4′-stearoylamino phenyl)-trimethylammonium methyl sulphuric acid) and lactose monohydrate until a uniform distribution of the constituents is obtained. A second preparation containing a disintegrating agent, such as maize starch, is added to the clioquinol mixture while being continuously stirred. Such a second preparation may be prepared by adding excess boiling water to a maize starch suspended in cold water. The final mixture is granulated and dried as above and mixed with maize starch and magnesium stearate and finally compressed into tablets in a standard apparatus.

A tablet may be coated or uncoated. An uncoated tablet may be scored. A coated tablet may be coated with sugar shellac film or other enteric coating agents.

Pharmaceutical formulations suitable for parenteral administration include sterile solutions or suspensions of the active constituents. An aqueous or oily carrier may be used. Such pharmaceutical carriers may be sterile Iiquids such as water and oils including those of petroleum animal, vegetable or synthetic origin such as peanut oil, soybean oil, mineral oil, sesame oil and the like. Formulations for parenteral administration also include a lyophilized powder comprising clioquinol and, optionally, further active constituents that is to be reconstituted by dissolving in a pharmaceutically acceptable carrier that dissolves the active constituents, e.g. an aqueous solution of carboxymethyl cellulose and lauryl sulphate.

Solutions can be dry, soluble products ready to be combined with a solvent just prior to use, suspensions ready for injections, dry, insoluble products ready to be combined with a vehicle just prior to use, emulsions, liquid concentrates ready for dilution prior to administration. The solubility of clioquinol as well as 8-hydroxyquinoline, 5,7-dichloro-8-hydroxy-quinoline, 5,7-dibromo-8-hydroxyquinoline can be increased by pH adjustment or the use of water miscible co-solvents or surfactants or complexing agents or the change of the dosage form to dispersed system (suspension, emulsion, liposome).

Aqueous parenteral solutions for intravenous or intramuscular or subcutaneous or intraspinal, or intracisternal or intrathecal or intraarterial pr intra-articular injection or infusion may be prepared by dilution to the desired concentration with an aqueous solvent or emulsifying agent, or the use of a cosolvents to increase solubility like water containing dissolved carboxymethylcellulose or polysorbate, such as polysorbate 80, ethyl oleate, Tween 20, or the like. Prior to the dissolution, the quinoline chelators may initially be pre-dissolved in an organic solvent, preferably an aprotic solvent like DMSO, DMF, and the like. Parental formulations are preferably made isotonic by adjusting with suitable electrolytes.

Clioquinol suspension for injection consists of insoluble solid particles dispersed in a liquid medium, with the solid particles accounting for 0.5-30% of the suspension. The vehicle may be aqueous, oil, or both. Excipients in injectable suspensions include antimicrobial preservatives, surfactants, dispersing or suspending agents, and buffers. Surfactants wet the suspended powders and provide acceptable syringeability while suspending agents modify the viscosity of the formulation.

Clioquinol emulsion for injection examples include oil-in-water sustained-release depot preparations, which are given intramuscularly.

When the pharmaceutical composition is a capsule, it may contain a liquid carrier, such as a fatty oil e.g. cacao butter.

Suitable pharmaceutical excipients include starch, glucose, lactose sucrose, gelatin, malt, rice, flour, chalk, silica gel, sodium stearate, glycerol monostearate, talc, sodium chloride, dried skim milk, glycerol, propylene, glycol, water, ethanol and the like. The compositions may be solutions suspensions emulsion tablets, pills, capsules, powders, sustained release formulations and the like. The composition may be formulated as a suppository, with traditional binders and carriers such as triglycerides.

In yet another embodiment, the clioquinol or phanquinone may be delivered in a controlled release system. In one embodiment, a pump may be used. In another embodiment, polymeric materials may be used. In yet another embodiment, a controlled release system may be placed in proximity of the therapeutic target, thus requiring only a fraction of the systemic dose.

In one embodiment of the pharmaceutical composition, clioquinol and the, optionally, further active constituents, are comprised as separate pharmaceutical entities. By way of example, one entity may comprise clioquinol and another entity may comprise phanquinone. The two entities, may be administered simultaneously or sequentially. For example, the entity comprising clioquinol can be administered, followed by phanquinone administered within a day, week, or month of clioquinol administration. If the two entities are administered sequentially, the entity comprising clioquinol is preferably administered for one to three weeks followed by a wash out period of one to four weeks. After the wash out period, the treatment may be repeated.

The pharmaceutical composition may be provided as a pack or kit comprising one or more entities containing one or more of the ingredients of the pharmaceutical compositions of the invention. Optionally, associated with such entities may be a notice in the form described by a governmental agency regulating the manufacture, use or sale of pharmaceuticals or biological products, which notice reflects approval by the agency of manufacture, use or sale for human administration.

Recently (Use of phanquinone in the treatment of Alzheimer's disease, PCT/IB/38/01095 Xilinas M et al, Jul. 17, 2003) we have undertaken extensive research for using phanquinone in the treatment of Alzheimer's disease. From experimental data it has been shown that phanquinone has the ability to chelate copper as well as zinc in the brain. The ability of copper chelation is three times more pronounced with phanquinone compared to clioquinol. It has also been shown that phanquinone possesses the same beneficial properties as clioquinol as to the abolishment of the amyloid formation and the resolubilisation of amyloid in the brain. In Example 2 the results of the effect of phanquinone and clioquinol on Zn- and Cu-induced A[beta] aggregation are shown.

In respect with the effect activity of clioquinol and phanquinone on zinc dependent proteins the substances were sent by the applicant to the National Institute of Health (NIH) that is running screening programmes in hepatitis B (HBV) and hepatitis C virus. The HBV research is run by the University of Georgetown (Dr. Brent Korba). The results with HBV were negative at this point because of cytotoxicity of the cell line used.

In research performed with the NIH collaboration with the Southern Research Institute (Dr. Victor Buckwold) the applicant has asked for the screening of clioquinol and phanquinone antiviral activity testing with hepatitis C virus. At this laboratory the effects established the effects of clioquinol and phanquinone with HCV were evaluated. The cell line used was Huh7 ET.

Phanquinone was evaluated using single high test concentration (2-micromolar) and an antiviral activity of 83.2% was found with a 10.5% cytotoxicity. In the retest with dose response curve the dose response of phanquinone showed no activity against HCV RNA replicons due to a high cytotoxicity of the Huh7 ET cell line.

Clioquinol in the same set of experiments showed to be potentially active with an antiviral activity of 82.3% and a selectivity index of over 1. In the retesting dose response clioquinol exhibited a selectivity index S150 over 4.8 and a S190 of 14.

The results that are presented in the Example 4 and conclude that clioquinol has a zinc protein activity on the HCV virus. As to phanquinone its potential antiviral activity cannot be exhibited yet because of strain specific cytotoxic effect on the Huh7 ET cell line used.

The data in Alzheimer's disease of clioquinol and phanquinone indicate and show their potent activity on the zinc and copper proteins such as beta amyloid. The same the activity of clioquinol on the Hepatitis C virus non structural metalloproteins

All documents cited in this application are included by reference. The invention is further illustrated with reference to the following examples which are provided for illustratory purposes and should not be considered limiting in any ways.

### EXAMPLES

### Example 1

### Preparation of a Pharmaceutical Composition Comprising Clioquinol

250 g of clioquinol were mixed with 200 g Sapamine@ (N-(4′-stearoyl amino-phenyl)-trimethylammonium methyl sulphuric acid) and 1025 g lactose mono-hydrate for a period of minutes. 5 g of boiling water was added in one go to a mixture of 0 g maize starch in 0 g cold water. The maize suspension, cooled to 40° C., was added to the clioquinol-containing powder mixture under continuous stirring. The mixture was granulated using a 2.5 mm sieve and desiccated for 18 hours at 40° C. The dry granules were mixed with 400 g maize starch and 20 g magnesium stearate. The final mixture was formulated into tablets having a diameter of 8.0 mm and a weight of 200 mg.

### Example 2

### Effect of Phanquinone and Clioquinol on Zn- and Cu-Induced Aβ Aggregation

A 5 mg/ml stock solution of Aβ (1-40) (delivered from Bachem (CH)) was freshly prepared before each experiment by dissolving the lyophilized peptide in 0.01 M HCl, followed by subsequent dilution 1.1 with 0.01 M NaOH to yield a neutral pH. Aliquot of Aβ (1-40) were diluted in PBS (pH 7.4) to 100 [mu]M and incubated at a total volume of 30 μ/l for 24 hours at room temperature. For co-incubation experiments, the indicated concentrations of metal ions and/or aliquot of test compounds were added. The test compounds were added to a final molar concentration of 10 μg/ml.

The amyloid formation was quantified by a thioflavin T fluorometric assay. Thioflavin binds specifically to amyloid and this introduces a shift in its emission spectrum and a fluorescent signal proportional to the amount of amyloid is formed. After incubation, Aβ (1-40) peptides were added to PBS (pH 6.0) and 3 μM thioflavin T in a final volume of 1 ml. Fluorescence was monitored at excitation 454 nm and emission 482 nm using a Fluoroscan II fluorometer (Molecular devices, UK). A time scan of fluorescence was performed and three values after the decay reached a plateau (around 5 minutes) were averaged after subtracting the background fluorescence of 3 μM thioflavin T. For co-incubation experiments, fluorescence of test compound alone was determined. Samples were run in triplicate.

Phanquinone and clioquinol were tested for their ability to prevent the aggregation of Aβ(1-40) into amyloid structures.

It was studied whether the two compounds had any effect on metal-ion catalysed Aβ aggregation, especially the aggregation caused by Zn and Cu.

At the tested concentration of 10 [μg/ml phanquinone reduced the Cu-induced aggregation by 50-60%, while the Zn-induced aggregation was only modest inhibited by approximately 10%. Unexpectedly, clioquinol showed the opposite tendency. Clioquinol reduced the Zn-induced aggregation of Aβ (1-40) by more than 60%, whereas the Cu-catalysed aggregation was reduced by approximately 30%. A pharmaceutical composition comprising phanquinone in combination with clioquinol may thus have a more widely usage than a pharmaceutical composition comprising one of the compounds alone.

### Example 3

### Effect of Phanquinone on Aβ [beta](25-35) Dose-Response in PC12 Cells

Aβ (25-35) was delivered by Bachem (CH) or Sigma (USA) and dissolved in phosphate buffered saline (PBS) at pH 7, 4, 2 hours prior to application. The neurotoxicity of Aβ is located in the sequence between amino acid residues 25 and 35 (Aβ (25-35)) and a decapeptide encompassing this region induces neural cell death equally potent as full length Aβ (1-40) (Yankner, Duffy L K, Kirschner D A: Neurotrophic and neurotoxic effects of amyloid β protein: reversal by tachykinin neuropeptides. Science 1990; 250:279-282).

Rat PC12 pheochromocytoma cells were grown in Dulbecco's modified Eagle's medium (DMEM) containing 1% penicillin-streptomycin, 5% fetal calf serum and 10% horse serum in humidified incubator with 5% CO₂.

PC12 cells were plated on 96-wells microtiter plates in 100 μl of the appropriate medium. After 24 hours the indicated concentrations of Aβ (25-35) peptide was added alone or together with phanquinone in the designated concentrations. Incubation continued for 24 hours. Following incubation, MTT reduction was measured using a commercially available assay according to the manufacturer's (Boehringer Mannheim) instructions. Assay values obtained by vehicle alone were defined as 100%.

MTT is a substrate for intracellular and plasma membrane oxidoreductases and has been widely used to measure reductions of cell redox activity. Reduction of the cell redox activity has been found to be an early indicator of Aβ mediated cell death (Shearman M S, Ragan C I, Iversen L L: Inhibition of PC12 cell redox activity is a specific, early indicator of the mechanism of β-amyloid-mediated cell death, Proc. Natl. Acad. Sci. USA 1994; 91:1470-1474).

To test the effect of phanquinone on Aβ (25-35) induced toxicity in PC12 cells, PC12 cells were exposed to Aβ (25-35) peptide concentrations ranging from 0 to 10 μM. In the absence of phanquinone (i.e. only vehicle added) Aβ (25-35) produced a dose-dependent inhibition of MTT reduction. Concentrations of Aβ (25-35) as low as 0.01 μM produced a significant reduction and at a concentration of Aβ (25-35) or above 0.1 μM the MTT reduction was reduced to a maximum level of about 50%.

In the presence of 10 μg phanquinone per ml the toxic effect of Aβ (25-35) was virtually abolished. Even at concentrations of Aβ as high as 1 μM, the presence of phanquinone completely counteracted the toxic effect of Aβ. Only at the highest concentration of Aβ (10 μM) there was a slight inhibition of MTT reduction by Aβ. This inhibition was, however moderate, only around 10% as compared to approximately 50% in the absence of phanquinone.

It will be obvious to a person skilled in the art that the invention thus described may be varied in many ways. Such variation are not to be regarded as a departure from the spirit and scope of the invention, and all such modifications, as would be obvious to a person skilled in the art, are intended to be included in the scope of the following claims.

### Example 4

### Screening Report Antiviral Activity Testing with Hepatitis C Virus Showing Activity on the Zinc Dependent Nonstructural HCV NS5A Protein

| | |
|---|---|
| ARB Number | 04-5868 |
| Drug Name | Phanquinone |
| Molecular Weight | 210.19 |
| Quantity Received | 4.95 mg |
| Researcher | Michel Xilinas |
| Company | No affiliation |
| Date Received | 19.5.2004 |
| Special Instructions | None |
| Assay Performed | HCV RNA Replicons |
| Assay Type | Single high test concentration* |
| Cell Type | Huh7 ET |
| Date Performed | 19.7.2004 |
| Antiviral Activity** | 83.2% |
| Cytotoxicity*** | 10.5% |
| Selectivity Index = | <1 |
| Comments | Toxic |

Southern Research Institute
*20 micromolar,
**luciferase, percent inhibition of virus control
***actively dividing cells, percent cell control

Screening Report Antiviral Activity Testing with Hepatitis C Virus Showing Activity on the Zinc Dependent Nonstructural HCV NS5A Protein

| | |
|---|---|
| ARB Number | 03-674 |
| Drug Name | Clioquinol |
| Molecular Weight | 305.5 |
| Quantity Received | 5 mg |
| Researcher | Michel Xilinas |
| Company | No affiliation |
| Date Received | 30.7.2004 |
| Special Instructions | Practically insoluble in water |
| Assay Performed | HCV RNA Replicons |
| Assay Type | Single high test concentration* |
| Cell Type | Huh7 ET |
| Date Performed | 20.9.2004 |
| Antiviral Activity** | 82.3% |
| Cytotoxicity*** | 73.3% |
| Selectivity Index = | >1 |
| Comments | Potentially active |

Southern Research Institute
*20 micromolar,
**luciferase, percent inhibition of virus control
***actively dividing cells, percent cell control

### Example 5

### Inhibition Study of MMPs by Clioquinol

An enzyme assay was conducted with five of the enzymes belonging to the MMP group. Specifically, the assay was conducted for MMP-1, MMP-2, MMP-3, MMP-7, and MMP-9 at various concentrations.

The MMP-1, MMP-3, and MMP-7 were initially pre-incubated in 60 min at 37EC and MMP-2 and MMP-9 were pre-incubated in 60 min at 25EC in an aqueous vehicle of 50 mM MOPS, 10 mM CaCl₂.2H₂O, 10 μM ZnCl₂, 0.050% Brij 35, pH 7.2 and a concentration of clioquinol of 100 μM. A test substrate of Mca-Pro-Leu-Gly-Leu-Dpa-Ala-Arg-NH₂ was subsequently added to obtain a concentration of 25 μM. MMP-1 was incubated for 2 hours at 37EC, MMP-2 was incubated for 3 hours at 25EC, MMP-3 was incubated for 90 min at 37EC. MMP-7 was incubated for 90 min at 37EC, and MMP-9 was incubated for 2 hours at 25 degrees Celsius. The activity of the enzymes was measured by fluorometric quantisation of Mca-Pro-Leu-Gly-OH. The results are indicated in the Table below.

| MMP | % Inhibition |
|---|---|
| MMP-1 | 12 |
| MMP-2 | 28 |
| MMP-3 | 7 |
| MMP-7 | 20 |
| MMP-9 | 19 |

The enzyme assay was repeated for MMP-2 except that a 10 and 100 times higher clioquinol concentration was used. At a clioquinol concentration of 1 mM the inhibition was 26% and at a clioquinol concentration of 10 mM the inhibition was measured to 101% the inhibition being highly dependent on clioquinol concentration

### Example 6

### Single Case Reports of Unwanted Effects, Overdosing, Teratogenicity and Interactions of Phanquinone

From the data contained in the single (spontaneous) case reports and from medical publications on phanquinone available till the withdrawal of the drug from the market the following conclusions may be drawn:

The most frequently reported unwanted effects in this review are those related to the gastrointestinal system. e.g. nausea. vomiting, abdominal pains (variously described) and diarrhea. The only other unwanted effects reported with relative frequency are irritability and headache. There is no evidence. however. that phanquinone has an adverse effect on the nervous system, the other reported symptoms are non specific in nature and infrequent. Nor there is any evidence that any system, other than the gastrointestinal is prone to be affected adversely by phanquinone with significant frequency.

The dosage of phanquinone employed was most often 200-400 mg/day for 7 to 10 days. When the daily dose was greater than 600 mg/day, or the duration of treatment was longer than two weeks, the incidence but not the severity of unwanted effects appeared to increase.

The authors of the publications on phanquinone have generally reported that the drug is well tolerated and that the unwanted effects observed were mild in degree and rarely necessitated discontinuation of treatment.

There have been no reports of deaths related to treatment with phanquinone and no reports of fetal malformations, drug interactions or carcinogenicity.

### Example 7

### Preparation of a Pharmaceutical Composition Comprising Phanquinone

250 g of phanquinone was mixed with 200 g Sapamine® (N-(4′-stearoyl amino-phenyl)-trimethylammonium methyl sulphuric acid) and 1025 g lactose mono-hydrate for a period of 5 minutes. 300 g of boiling water was added in one go to a mixture of 100 g maize starch in 100 g cold water. The maize suspension, cooled to 40° C. was added to the phanquinone containing powder mixture under continuous stirring. The mixture was granulated using a 2.5 mm sieve and desiccated for 18 hours at 40° C. The dry granules were mixed with 400 g maize starch and 20 g magnesium stearate. The final mixture was formulated into tablets having a diameter of 8.0 mm and a weight of 200 mg.

Various publications are cited herein, the disclosure of which are incorporated by reference in their entireties. The invention being thus described, it will be obvious that the same may be varied in many ways. Such variations are not to be considered a departure form the spirit and scope of the invention, and all such modifications, as would be obvious to a person skilled in the art, are intended to be included in the scope of the claims.

1-24. (canceled)

25. A method for treating a disease or condition caused by a pathological increase in production of, or an imbalance or dysregulation of the activity of, (i) a zinc-dependent protein, (ii) a copper-dependent protein, (iii) a zinc-containing protein or (iv) a copper-containing protein, the method comprising administering to a subject in need thereof an effective amount of a pharmaceutical composition which comprises of one or more chelators which is zinc-specific, copper-specific or zinc- and copper-specific, and which chelator modulates the production, balance or regulation of said protein.
wherein the disease or condition is cancer or a tumor, an acute coronary syndrome, a macular disease, diabetes or an inflammatory disease,
which inflammatory disease is rheumatoid arthritis, osteoarthritis, inflammatory bowel disease, Sjögren's syndrome, ankylosing spondylitis, Behcet's syndrome, vasculitis, sarcoidosis, psoriatic arthritis or polychondritis.

26. The method according to claim 25, wherein said one or more chelators is selected from the group consisting of:
clioquinol; phanquinone; 8-hydroxyquinoline; 5,7-dichloro-8-hydroxyquinoline; 5,7-dibromo-8-hydroxyquinoline; 2-methyl-8-hydroxyquinoline; 2-methyl-8-hydroxyquinaldine; 5,7-dichloro-2-methyl-8-hydroxyquinoline; 5,7-dichloro-8-hydroxyquinaldine, 5-methyl-oxine (5-methyl-8-hydroxyquinoline); 2-mercaptopyridine-N-oxide; 5-formyl-8-hydroxyquinoline; 5-iodo-8-hydroxyquinoline; 5-fluoro-8-hydroxy-quinoline; 5-acetyl-8-hydroxyquinoline; 5-methoxymethyl-8-hydroxyquinoline; ethyl-5-(8-hydroxyquinolyl)acetate; methyl-5(8-hydroxyquinolyl)acetate; ethyl-5-(8-hydroxyquinolyl)acetate; 2,7,8-trihydroxyquinoline; a 4-hydroxyquinoline-3-carboxylate; a 5-phenylethyl-4-hydroxyquinoline-3-carboxylic acid; dibromo-8-benzoyloxyquinoldine; an arylglyoxal N-7-amino-5-substituted 8-hydroxyquinoline hemiacetal; a 5-phenylglyoxylidenamin-8-hydroxyquinoline; α,α′-dipyridyl-8-hydroxy-quinoline; 2,2′,2″-tripyridine; 2,6-dihydroxyquinoline; 5-formyl-1-methoxycarbonyl-4,6,8-trihydroxyphenazine; a 5- or 7-methylthio-8-hydroxyquinoline derivative; a 4-hydroxyquinoline-2-carboxylic acid; a 3-hydroxyquinoline-2-carboxylic acid; a 5-phenylethyl-4-hydroxyquinoline-3-carboxylic acid; a 2-n-alky-1-4-hydroxyquinoline derivate; O-acetyl-8-hydroxyquinoline; S-acetyl-8-mercaptoquinoline; 5,7 dibromo-8-benzoyloxyquinoldine; a 5-phenylglyoxylidenamin-8-hydroxyquinoline; ethyl 6,7-di-isobutoxy-4-hydroxyquinoline-3-carboxylate; 5-chloro-8-hydroxyquinoline; a 5-chloro-8-hydroxy-quinoline (chloroxine) carboxylic acid ester; m-phenylenediamine; 1aH-oxazirino[2,3-aquinoline-1a-carbonitrile or a substituted derivative thereof to a corresponding 3-hydroxyquinoline derivative; 6,7-dimethoxy-4-hydroxyquinoline hydrochloride; 4-nitroquinoline 1-oxide; nitroxoline; 8-hydroxyquinoline-7-carboxylic acid; hydroxy-quinoline sulfate; chlorohydroxyquinoline; 8-hydroxyquinoline-5-sulfonic acid; a derivative of 1,2,3,4-tetrahydroquinoline; 2-heptyl-4-hydroxyquinoline-N-oxide; 2-n-heptyl-4-hydroxyquinoline-N-oxide; a 4-hydroxyquinoline-3-carboxylic acid that is 7-substituted with benzyloxy, phenethyloxy or phenoxyethyloxy; 6-hydroxyquinoline; 6-nitroquinoline; 8-nitroquinoline; 6-methylquinoline; 8-methylquinoline; 8-hydroxy-quinoline; 5,7-dibromoquinoline; 2-n-nonyl-4-hydroxy[3-3H]quinoline; 2n-nonyl-4-hydroxyquinoline-N-oxide; an 8-hydroxyquinoline-5-sulfonic acid; a 2,2′,2″-terpyridine complex; 8-hydroxyquinoline-5-sulfonic acid; 5-nitro-8-hydroxyquinoline; 2-mercaptoquinoline-N-oxide; 5,6-benzoquinoline; 5-methyl-8-hydroxyquinoline-4-hydroxy-quinoline; 2-n-alkyl-4-hydroxyquinoline; ethylenediaminetetraacetic acid; O-phenanthroline; 1,10-phenanthroline; 2,9-dimethyl-1,10-phenanthroline; (2′-hydroxyphenyl)pyridine; rhodotorulic acid; mycobactin P; 8-hydroxyquinoline-7-carboxylate; a 5-substituted 8-hydroxyquinoline; 5-chloro-8-hydroxyquinoline; 4-hydroxyquinoline; a 6-substituted-4-hydroxyquinoline-5-carboxylic acid; a 7-substituted-4-hydroxyquinoline-5-carboxylic acid; a Δ-substituted-4-hydroxyquinoline-5-carboxylic acids; 5-7-dibromo-8-hydroxyquinoline; 2,2′-bipyridine; an 8-hydroxyquinoline ester; a halogen derivative of salicylanilide; batimastat; a hydroxamic acid and a fenamate.

27. A method according to claim 25 wherein the protein is selected from the group consisting of epidermal growth factor, fibroblast growth factor, granulocyte platelet derived factor, colony stimulating factor, tumor necrosis factor α, vascular endothelial growth factor, zinc/copper superoxide dismutase, cathepsin, a gelatinase, stromelysin, urokinase-type plasminogen activator, a zinc-dependent protease, a zinc-dependent endopeptidase, interleukin-1, interleukin-6, interleukin-8, nitric oxide synthase, β-amyloid and phospholipase.

28. The method according to claim 25 wherein the disease or condition is a tumor, primary or metastatic cancer, tumor-induced neoangiogenesis, or a myeloproliferative disorder.

29. The method according to claim 28 wherein the cancer or tumor is lung, colon, rectal gastric, breast, prostate, uterine, ovarian, urinary tract, oral, pancreatic, melanoma or other skin cancer or tumor.

30. The method according to claim 28 wherein the cancer or myeloproliferative disorder is lymphoma, leukemia or myeloid metaplasia.
